(19) United States
(12) Patent Application Publication
(10) Pub. No.: US 2009/0275622 A1
(43) Pub. Date: Nov. 5, 2009

(54) NIZATIDINE FORMULATIONS

(76) Inventors: Prasoona Linga, Nalgonda (IN); Arunkumar Haridas Mantri, Hyderabad (IN); Shrikant Vitthalrao Dhoke, Nagpur (IN); Raviraj Sukumar Pillai, Hyderabad (IN)

Correspondence Address: DR. REDDY''S LABORATORIES, INC., 200 SOMERSET CORPORATE BLVD, SEVENTH FLOOR, BRIDGEWATER, NJ, 08807-2862, US

(21) Appl. No.: 12/433,065

(22) Filed: Apr. 30, 2009

Related U.S. Application Data
(60) Provisional application No. 61/081,834, filed on Jul. 18, 2008.

(30) Foreign Application Priority Data
Apr. 30, 2008 (IN) 1074/CHE/2008

(51) Int. Cl. A61K 31/426; A61P 1/04

(52) U.S. Cl. 514/365

(57) ABSTRACT

Pharmaceutical formulations comprising nizatidine or salts thereof. Further the invention relates to liquid pharmaceutical formulations comprising nizatidine or salts thereof.

### INTRODUCTION

The present invention relates to pharmaceutical formulations comprising nizatidine or salts thereof. Further the invention relates to liquid pharmaceutical formulations comprising nizatidine or salts thereof.

Nizatidine has a chemical name N-[2-[[[2-[(dimethylamino)methyl]-4-thiazolyl]methyl]thio]ethyl]-N′-methyl-2-nitro-1,1-ethenediamine. It has structural Formula I.

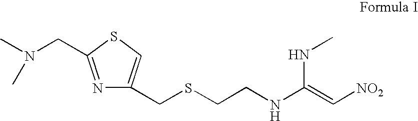

Nizatidine is a competitive, reversible inhibitor of histamine at the histamine H₂ receptors, particularly those in the gastric parietal cells. By inhibiting the action of histamine on stomach cells, nizatidine reduces stomach acid production. Nizatidine has no demonstrable antiandrogenic action. It has been demonstrated that treatment with a reduced dose of nizatidine is effective as maintenance therapy following healing of active duodenal ulcers.

Nizatidine, as a histamine H₂ receptor antagonist, is useful as an anti-ulcer agent. It is prescribed for the treatment of active duodenal ulcer, maintenance therapy of endoscopically diagnosed esophagitis including erosive and ulcerative esophagitis and also in the treatment of active benign gastric ulcer.

Products containing the drug are commercially available in the market as AXID® oral solution, containing 15 mg/mL of nizatidine for oral administration, from Braintree Laboratories Inc., and as AXID® capsules, containing 150 mg and 300 mg of nizatidine, from SmithKline Beecham.

The inactive ingredients in AXID oral solution are methylparaben, propylparaben, glycerin, sodium alginate, purified water, sodium chloride, saccharin sodium, sodium citrate dihydrate, citric acid anhydrous, sucrose, bubble gum flavor, artificial sweetness enhancer, and sodium hydroxide.

Nizatidine is white to buff crystalline solid that is soluble in water. Nizatidine has a bitter taste and mild sulphur-like odor. There is a need for producing taste and odor masked formulations, to obtain formulations that are palatable for oral administration.

U.S. Pat. No. 4,375,547 discloses the nizatidine compound. U.S. Pat. No. 6,930,119 discloses a nizatidine oral solution having sodium alginate as a taste masking agent, wherein the weight ratio of nizatidine to the alginate is from 1:0.1 to 1:0.6.

U.S. Pat. No. 6,555,139, and U.S. Patent Application Publication Nos. 2006/0105045, 2002/0150616, and 2008/0075784, disclose pharmaceutical formulations comprising cyclodextrins.

There remains a need for the development of pharmaceutical formulations comprising nizatidine with improved taste masked properties and palatability.

### SUMMARY

An aspect of the present invention relates to pharmaceutical formulations comprising nizatidine or salts thereof. A further aspect relates to liquid pharmaceutical formulations comprising nizatidine or salts thereof.

In an embodiment the invention relates to pharmaceutical formulations comprising nizatidine or salts thereof, wherein the taste of nizatidine or a salt thereof is masked.

In an embodiment the invention relates to pharmaceutical solution formulations suitable for oral administration, comprising nizatidine or salts thereof and one or more taste-masking and/or taste-enhancing agents. Embodiments of the invention may include formulations having pH values about 6 to about 7.

In an embodiment the invention includes pharmaceutical solution formulations comprising nizatidine or salts thereof, and cyclodextrins or their derivatives.

In an embodiment the invention includes pharmaceutical solution formulations comprising nizatidine or salts thereof, and cyclodextrins or their derivatives, wherein weight ratios of nizatidine or salts thereof to the taste-masking agents is about 1:0.01 to about 1:1-5.

In an embodiment the invention relates to pharmaceutical solution formulations comprising nizatidine or salts thereof, and xanthan gum.

In an embodiment the invention relates to pharmaceutical solution formulations comprising nizatidine or salts thereof, and hydroxyethyl cellulose.

In an embodiment the invention relates to pharmaceutical solution formulations comprising nizatidine or salts thereof, and hydroxypropyl methylcellulose.

In an embodiment the invention relates to pharmaceutical solution formulation comprising nizatidine or salts thereof, and sodium alginate, with the proviso that the weight ratios of nizatidine or salts thereof to sodium alginate is in the range of about 1:0.8 to about 1:2, or to about 1:5.

In an embodiment the invention includes processes for preparing pharmaceutical formulations comprising nizatidine that assist in the effective delivery of nizatidine, and methods of using such formulations for the treatment of duodenal ulcers.

### DETAILED DESCRIPTION

The present invention relates to pharmaceutical formulations comprising nizatidine or salts thereof. Further the invention relates to liquid pharmaceutical formulations comprising nizatidine or salts thereof.

Liquid pharmaceutical formulations for oral administration frequently contain about 0.5 to about 30 mg/mL of nizatidine or a salt thereof, or about 15 mg/mL of nizatidine.

The term “pharmaceutical formulation” as used herein refers to solid or liquid pharmaceutical formulations comprising nizatidine or salts thereof with one or more additional pharmaceutically acceptable excipients, such as those required to mask the taste of nizatidine or salts thereof, for the effective delivery of nizatidine or salts thereof.

The term “palatable” as used here in refers to a formulation being sufficiently agreeable in taste to be ingested.

The term “taste-masking” and/or “taste-enhancing” as used herein refers to any agent which either suppresses the bitter taste of a drug or brings about an improvement in the taste so that it is palatable for oral administration.

The term “inclusion complex” as used herein refers to a complex in which one chemical compound (the “host”) forms a cavity in which molecules of a second “guest” compound can be located. The definition of inclusion compounds is very broad, extending to channels formed between molecules in a crystal lattice into which guest molecules can fit.

Taste masking and/or taste enhancing properties may be achieved by various methods such as complexation with alginates, inclusion complexation with cyclodextrins or their derivatives, use of viscosity increasing agents such as gums or cellulose based polymers, use of oily formulations, use of self-emulsifying drug delivery systems, formation of co-precipitates with hydrophilic polymers, co-milling with hydrophilic excipients, co-precipitates or solid dispersions, premixes, use of amorphous or alternate crystalline forms, and the like, including combinations thereof.

In an embodiment the invention relates to pharmaceutical solution formulations suitable for oral administration, comprising nizatidine or a salt thereof and one or more taste-masking and/or taste-enhancing agents. In an embodiment the invention includes pharmaceutical solution formulations comprising nizatidine or salts thereof, and cyclodextrins or their derivatives.

In an embodiment the invention includes pharmaceutical solution formulations comprising nizatidine, or salts thereof, and cyclodextrins or their derivatives, wherein weight ratios of nizatidine to cyclodextrin or derivatives are in the range of about 1:0.01 to about 1:10, or from about 1:0.25 to about 1:5.

As used herein “cyclodextrin” refers to the natural cyclodextrins, α-cyclodextrin, β-cyclodextrin, and γ-cyclodextrin, and their respective derivatives. The formation of inclusion compounds with cyclodextrins greatly modifies the physical and chemical properties of the guest molecule, mostly in terms of water solubility and masking taste of the molecule. Cyclodextrins are used for inclusion complex formation, and are capable of masking unpleasant tastes of drugs by decreasing the amount of drug particles exposed to the taste buds, thereby reducing the perception of unpleasant taste.

The cyclodextrins useful in the present invention include the natural occurring cyclodextrins and their derivatives. The natural cyclodextrins include α-cyclodextrin, β-cyclodextrin and γ-cyclodextrin. Derivatives are typically prepared by modifying the hydroxyl groups located on the exterior, or hydrophilic side, of the cyclodextrin. The modifications can be made to increase the aqueous solubility and the stability of the complex and can modify the physical characteristics of the complex including the formation and dissociation of the complex. The types and the degree of modification, as well as their preparation, are well known in the art. Any of the natural cyclodextrins can be derivatized, such as derivatives of β-cyclodextrin. Cyclodextrin derivatives include alkylated cyclodextrins, comprising methyl-, dimethyl-, trimethyl- and ethyl-β-cyclodextrins; hydroxyalkylated cyclodextrins, including hydroxyethyl-, hydroxypropyl-, and dihydroxypropyl-β-cyclodextrin; ethylcarboxymethyl cyclodextrins; sulfate, sulfonate and sulfoalkyl cyclodextrins, such as β-cyclodextrin sulfate, β-cyclodextrin sulfonate, and β-cyclodextrin sulfobutyl ether; as well as polymeric cyclodextrins. Other cyclodextrin derivatives can be made by substitution of the hydroxy groups with saccharides, such as glucosyl- and maltosyl-β-cyclodextrin. Other cyclodextrins include the naturally occurring cyclodextrins, methyl-β-cyclodextrin, dimethyl-β-cyclodextrin, trimethyl-β-cyclodextrin, 2-hydroxymethyl-β-cyclodextrin, hydroxyethyl-β-cyclodextrin, 2-hydroxypropyl-β-cyclodextrin, 3-hydroxypropyl-β-cyclodextrin, β-cyclodextrin sulfate, β-cyclodextrin sulfonate, or β-cyclodextrin sulfobutyl ether. Any of the above cyclodextrins or their derivatives or polymers prepared from them can be used for preparation of formulations of the invention, either alone or in the form of mixtures of one or more cyclodextrins.

Commercially available cyclodextrins may be used, such as are available from any of the commercial suppliers including, for example, Cargill, Roquette, Aldrich Chemical Company, Milwaukee, Wis. USA, and Wacker Chemicals, New Canaan, Conn. USA, or may be synthesized by any of the processes known in the art for the synthesis of cyclodextrins and their derivatives.

In embodiments, the complexation is complete or partial, wherein nizatidine or a salt thereof and the cyclodextrin exist together in intimate contact and result in a clear solution containing the active agent.

In an embodiment the invention includes use of hydroxypropyl β-cyclodextrin (HPβCD) for complexation with nizatidine or salt thereof.

Embodiments of the invention may include nizatidine or a salt thereof and a cyclodextrin compound, such as HPβCD, being present in weight ratios of about 1:0.01 to about 1:1-5.

It is desirable that the nizatidine or salt thereof may be present as an inclusion complex with very little or no free nizatidine in the solution formulation.

Various methods are known in the art to prepare inclusion complexes with cyclodextrins such as the solution method, co-precipitation method, slurry method, kneading method, and grinding method. See T. Loftsson, “Pharmaceutical applications of β-cyclodextrin,” Pharmaceutical Technology, Vol. 12, pp. 41-50, 1999.

In the solution method, the drug, either as a solid or in a solution, is added to a solution containing an excess amount of cyclodextrin. It is also possible to add an excess of the drug to an aqueous cyclodextrin solution. The mixture is agitated, and may optionally be heated, until equilibrium is reached, which may take several hours or several days. The equilibrated solution is then filtered or centrifuged to give a clear solution of the drug-cyclodextrin complex. The clear solution can be directly administered to a subject, or a solid complex can be obtained by removal of the water by evaporation (such as spray-drying), sublimation (such as lyophilization), or other drying means well known in the art. A solid complex may also be obtained by the precipitation method. Often, the cyclodextrin complexes will precipitate upon cooling of the solution. Otherwise, a solvent in which the complex has minimal solubility, typically an organic solvent, is used to precipitate the solid complex. The precipitate containing the complex can then be filtered or centrifuged to obtain a solid drug-cyclodextrin complex. A generally less effective method of preparing a solid complex mixture is to grind a dry mixture of the drug and cyclodextrin in a sealed container, which is then gently heated to a temperature between 60 and 140° C.

The amount of free drug present in the solution formulation will be determined by the amount and type of the cyclodextrin, the complexation capacity of the cyclodextrin selected, the process utilized to prepare the liquid formulation, and other parameters known to a person skilled in the art.

Any method may be used for the preparation of the inclusion complexes of the invention including but not limited to the methods described above.

Sometimes suspending agents may be used for taste masking. Various examples of suspending agents include xanthan gum, alginates, alginic acid or sodium alginate, carboxymethyl celluloses, hydroxyethyl celluloses, hydroxypropyl methylcelluloses, methylcelluloses, microcrystalline celluloses, carboxymethyl cellulose blends, glycerin, carageenan, tragacanth, guar gum, pectin, and any mixtures thereof.

In an embodiment the invention relates to pharmaceutical solution formulations comprising nizatidine or a salt thereof, and xanthan gum.

Embodiments of the invention may include nizatidine or salt thereof and the xanthan gum being present in weight ratios of about 1:0.01 to about 1:1-5.

In an embodiment the invention relates to pharmaceutical solution formulations comprising nizatidine or a salt thereof, and hydroxyethyl cellulose.

Embodiments of the invention may include nizatidine or a salt thereof and hydroxyethyl cellulose being present in weight ratios of about 1:0.01 to about 1:1-5.

In an embodiment the invention relates to pharmaceutical solution formulations comprising nizatidine or salts thereof, and sodium alginate, with the proviso that the weight ratios of nizatidine or a salt thereof to sodium alginate are in the range of about 1:0.8 to about 1:2, or about 1:5.

It is desirable, though not essential, that the nizatidine have a uniform particle size distribution, which will aid in the processing of the formulation.

The solvent media useful in the preparation of the inclusion complexes include but are not limited to water, methanol, ethanol, acidified ethanol, acetone, diacetone, polyols, polyethers, oils, esters, alkyl ketones, acetonitrile, methylene chloride, isopropyl alcohol, butyl alcohol, methyl acetate, ethyl acetate, isopropyl acetate, castor oil, ethylene glycol monoethyl ether, diethylene glycol monobutyl ether, diethylene glycol monoethyl ether, dimethylsulphoxide, N,N-dimethylformamide, tetrahydrofuran, and any mixtures thereof.

In an embodiment of the invention, water, aqueous solutions, or mixtures of water with a water-miscible organic solvent, is used for the preparation of cyclodextrin inclusion complexes. Any solvent medium is acceptable for the preparation of inclusion complexes of the invention as long as the active agent is soluble or dispersible in the medium, the cyclodextrin is soluble in the medium, and the medium is not chemically detrimental to the active or the complex formed.

The processes of preparing the solubility-enhanced form can further involve the addition of a pharmaceutically acceptable bulking agent, and addition of complexation enhancers as desired.

The inclusion complexes may be prepared using the cyclodextrin compound and nizatidine, in the presence of a solvent medium and other pharmaceutically acceptable excipients may be added to aid the preparation of the formulations. Such pharmaceutically acceptable excipients can include for example wetting agents, pH modulators, complexation enhancers, and the like.

Complexation enhancers may be in the form of surfactants, alkalizing agents, acidifying agents, and solubilizers. They may be used either alone or in combination.

Taste-enhancing agents comprise one or more acidifying or alkalizing agents. Embodiments of the of taste-enhancing agents include one or more of citric acid, sodium citrate dihydrate, saccharin sodium, sodium chloride, sodium hydroxide, sodium carbonate, sodium bicarbonate, artificial sweetness enhancers, and any mixtures thereof.

Suitable acidifying agents include, but are not limited to, hydrochloric acid, carbonic acid, phosphoric acid, histidine hydrochloride, glycine hydrochloride, citric acid, and any mixtures thereof.

Suitable alkalizing agents include, but are not limited to organic amines such as meglumine, tromethamine, triethanolamine, diethanolamine, etc.; inorganic alkali metal compounds such as for example sodium hydroxide, sodium carbonate, sodium bicarbonate and the like; amino acids such as alanine, isoleucine, leucine, methionine, phenylalanine, proline, tryptophan, valine, asparagine, cysteine, glutamine, glycine, serine, threonine, tyrosine, aspartic acid, glutamic acid, arginine, histidine, and lysine; and mixtures thereof.

In an embodiment, the invention includes complexation enhancers that are either acidifying or alkalizing agents, as discussed herein.

Surfactants include, but are not limited to, sodium lauryl sulfate, polysorbate 80, poloxamer 188, poloxamer 407, sodium carboxymethylcellulose, hydrogenated oil, polyoxyethylene glycol, polyoxypropylene glycol, polyoxyethylene sorbitan fatty acid esters, polyglycolized glycerides available commercially such as GELUCIRE™ 40/14, GELUCIRE 42/12, and GELUCIRE 50/13, Vitamin E TGPS, etc.

Emulsifying agents include, but are not limited to, a wide variety of cationic, anionic, zwitterionic, and amphoteric surfactants known in the art. Non-limiting examples of anionic emulsifying agents include the alkoyl isethionates, alkyl and alkyl ether sulfates and salts thereof, alkyl and alkyl ether phosphates and salts thereof, alkyl methyl taurates, and soaps such as for example alkali metal salts including sodium or potassium salts of long chain fatty acids.

Examples of amphoteric and zwitterionic emulsifying agents include, but are not limited to, carboxy, sulfonate, sulfate, phosphate, or phosphonate compounds. Other examples are alkyl iminoacetates and iminodialkanoates and aminoalkanoates, imidazolinium and ammonium derivatives, betaines, sultaines, hydroxysultaines, alkyl sarcosinates and alkanoyl sarcosinates, and the like.

Examples of other suitable emulsifying agents include disodium cocoamphodiacetate, oxyethylenated glyceryl cocoate (7 EO), PEG-20 hexadecenyl succinate, PEG-15 stearyl ether, ricinoleic monoethanolamide monosulfosuccinate salts, oxyethylenated hydrogenated ricinoleic triglyceride, poloxamers, non-solid fatty substances such as sesame oil, almond oil, apricot stone oil, sunflower oil, octoxyglyceryl palmitate (or 2-ethylhexyl glyceryl ether palmitate), octoxyglyceryl behenate (or 2-ethylhexyl glyceryl ether behenate), dioctyl adipate, tartrates of branched dialcohols, and the like.

Other useful non-ionic emulsifying agents include alkylene oxide esters of fatty acids, alkylene oxide diesters of fatty acids alkylene oxide ethers of fatty alcohols, alkylene oxide esters, and the like.

In an embodiment the invention includes the use of emulsifiers such as sodium lauryl sulfate, polysorbate 80, polyglycolized glycerides available commercially in grades such as GELUCIRE 40/14, GELUCIRE 42/12, and GELUCIRE 50/13, Vitamin E TPGS, and the like.

In one aspect the pH of the formulations is optionally adjusted to a desired pH range. Any pH is acceptable as long as it is not detrimental to the chemical stability of nizatidine or salts thereof. Any of the acidifying or alkalizing agents mentioned above can be used for adjusting the pH to the desired range. Embodiments of the invention include solution formulations having pH values about 6 to about 7.

Further the process involves mixing of the components to form a clear solution. Any means of mixing is acceptable as long as it provides a clear solution of the nizatidine in the aqueous medium. Such mixing means could include for example overhead stirrers, homogenizers, static mixers, sonicators and the like. The duration of mixing will be decided based on parameters such as concentration to be achieved, the temperature of the dispersion, the types of excipients, the mixing means, the particle size of the nizatidine or salt thereof and such other parameters known to a person skilled in the art.

The solution obtained as described above may be filtered to remove extraneous material or undissolved drug substance to prevent these from getting into the final product. Any filter medium may be chosen such as for example different grades of membrane filters, sintered glass filters and the like. The filtered solution may optionally be subjected to evaporation of the solvent medium to recover a dry product. Any method of solvent evaporation or drying is acceptable as long as it is not detrimental to the chemical stability of the drug as well as the solubilizing formulation. Such methods can include for example tray drying, vacuum drying, spray drying, spray coating, lyophilization, microwave drying and the like without limitation. Two or more methods can be used sequentially to ensure completeness of removal of the solvent medium or to achieve desirable bulk properties of the dried solubilizing formulations.

Aspects of the invention provide processes for preparing formulations comprising nizatidine or salt thereof, wherein an embodiment of a process comprises:

a) Heating water and dissolving a sweetener.

b) Dissolving/dispersing a preservative in a solvent.

c) Dissolving/dispersing a complexing agent in water.

d) Dissolving drug in the solution of step c).

e) Dissolving remaining excipients in water.

f) Adding step b), d) and e) materials to step a) and mixing well.

g) Optionally adding flavors to step f).

h) Diluting to the desired volume with water.

In embodiments the invention includes processes for preparing pharmaceutical formulations comprising nizatidine or salt thereof that assist in the effective delivery of nizatidine and methods of using such formulations for the treatment of duodenal ulcers.

There are several impurities reported for nizatidine in European Pharmacopeia 5.0, at pages 2112-2113, including impurities A, B, C, D, E, F, G, H, I, J, and K:

A has a chemical name N,N′-dimethyl-2-nitroethene-1,1-diamine.

B has a chemical name (EZ)-N-methyl-1-(methylsulphanyl)-2-nitroethen-1-amine.

C has a chemical name (EZ)-N-[2-[[[2-[(dimethylamino)methyl]thiazol-4-yl]methyl]sulphinyl]ethyl]-N′-methyl-2-nitroethene-1,1-diamine.

D has a chemical name 2-[[[2-[(dimethylamino)methyl]thiazol-4-yl]methyl]sulphanyl]ethanamine.

E has a chemical name N-[2-[[[2-[(dimethylamino)methyl]thiazol-4-yl]methyl]sulphanyl]ethyl]-2-nitroacetamide.

F has a chemical name (EZ)-N¹,N¹′-[thiazole-2,4-diylbis(methylenesulphanediylethylene)]bis(N′-methyl-2-nitroethene-1,1-diamine).

G has a chemical name N,N′-bis[2-[[[2-[(dimethylamino)methyl]thiazol-4-yl]methyl]sulphanyl]ethyl]2-nitroethene-1,1-diamine.

H has a chemical name 2-(dimethylamino)thioacetamide.

I has a chemical name N-[2-[[[2-[(dimethylamino)methyl]thiazol-4-yl]methyl]sulphanyl]ethyl]-N′-methylurea.

J has a chemical name [2-[(dimethylamino)methyl]thiazol-4-yl]methanol.

K has a chemical name 3-(methlyamino)-5,6-dihydro-2H-1,4-thiazin-2-one oxime.

In addition, three impurities (denoted herein as impurities X, Y and Z) have been identified in formulations at the time of preparation, as well as during stability studies.

Impurity X has a chemical name (E)-3-(2-((dimethylamino)methyl)thiazol-4-yl)-N-(2-((2-((dimethylamino)methyl)thiazol-4-yl)methylthio)ethyl)-N-methyl-2-nitroprop-1-ene-1,1-diamine, and is represented by Formula II.

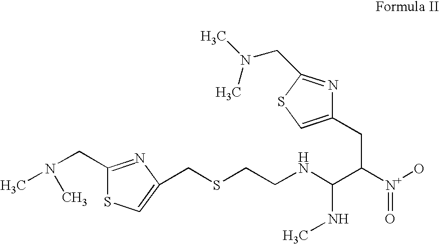

Impurity Y has a chemical name 6-(2-((2-((dimethylamino)methyl)thiazol-4-yl)methylthio)ethylamino)-1-methyl-5-nitro-4-phenyl-1,2,3,4-tetrahydropyridin-2-ol, and is represented by Formula III.

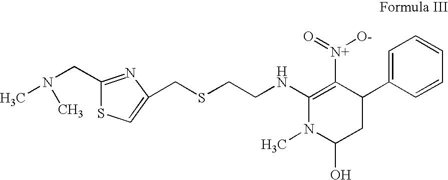

Impurity Z has a chemical name 2-(2-((2-((dimethylamino)methyl)thiazol-4-yl)methylthio)ethylamino)-1-methyl-4-phenylpyridinium, and is represented by Formula IV.

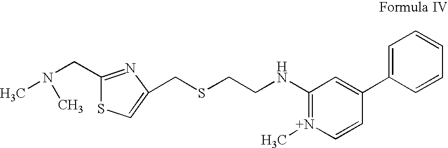

In an aspect, the invention relates to analytical methods for analyses of nizatidine-related impurities using high performance liquid chromatography (HPLC), wherein an embodiment of a method utilizes the conditions below.

Buffer solution: 5.9 g of ammonium acetate in 760 mL of milli Q water, 1 mL of diethylamine is added, and pH is adjusted to 7.5 with acetic acid.

Mobile phase A: Buffer and methanol in the volume ratio of 9:1.

Mobile phase B: Methanol and buffer in the volume ratio of 9:1.

Diluent: Buffer and methanol in the volume ratio of 9:1.

Chromatographic system:

a) Liquid chromatograph equipped with a 254 nm UV detector.

b) Column: 4.6 mm×250 mm Inertsil ODS-3V, 5 μm.

c) Column temperature: 45° C.

d) Flow rate: 1 mL per minute.

e) Injection volume: 25 μL.

f) Run time: 95 minutes.

g) Mobile phase gradient program:

| | % of Mobile | % of Mobile |
|---|---|---|
| Minutes | Phase A | Phase B |
| 0 | 100 | 0 |
| 25 | 85 | 15 |
| 30 | 75 | 25 |
| 45 | 80 | 20 |
| 55 | 65 | 35 |
| 60 | 65 | 35 |
| 75 | 50 | 50 |
| 80 | 25 | 75 |
| 85 | 25 | 75 |
| 87 | 100 | 0 |
| 95 | 100 | 0 |

Preparation of test sample: 2 g of nizatidine oral solution is transferred into a 25 mL volumetric flask, about 17 mL of diluent are added, the mixture is sonicated for about 10 minutes with intermediate shaking, and then diluent is added to make the 25 mL volume.

Representative relative retention times (RRT, where nizatidine=1) are tabulated below.

| Impurity | RRT |
|---|---|
| Impurity X | 0.95 |
| Impurity Y | 1.72 |
| Impurity Z | 1.78 |

In embodiments the invention includes methods of treating duodenal ulcer or endoscopically diagnosed esophagitis, including erosive and ulcerative esophagitis, and associated heartburn due to GERD or active benign gastric ulcer, comprising administering a pharmaceutical solution formulation.

Certain specific aspects and embodiments of the invention will be further described in the following examples, which are provided only for purposes of illustration and are not intended to limit the scope of the invention in any manner.

### EXAMPLE 1

### Pharmaceutical Solution Formulations of Nizatidine 15 mg/mL with Hydroxypropyl-β-Cyclodextrin

Grams/500 mL

| Ingredient | A | B |
|---|---|---|
| Nizatidine | 7.5 | 7.5 |
| Hydroxypropyl-β-cyclodextrin | 15 | 7.5 |
| Citric acid (anhydrous) | 1.05 | 1.05 |
| Sodium citrate dihydrate | 0.03 | 0.03 |
| Propylparaben | 0.1 | 0.1 |
| Methylparaben | 0.9 | 0.9 |
| Glycerin | 45 | 45. |
| Sodium chloride | 1.5 | 1.5 |
| Sodium saccharin | 2.5 | 2.5 |
| Granulated sucrose | 150 | — |
| Sucrose | — | 200 |
| Bubble gum flavor | 0.3 | 0.3 |
| Mint flavor | 0.25 | 0.25 |
| Prosweet# | 5 | 5 |
| Ethanol | 10 | 5 |
| Water | q.s. to 500 mL | q.s. to 500 mL |

#Prosweet is a proprietary blend of natural sweeteners, obtained from Virginia Dare.

Formulation pH range: 6-7.

Manufacturing process:

1) Heat 100 mL of water to about 70-90° C., add sucrose, and stir to dissolve.

2) Dissolve methylparaben and propylparaben in half of the quantity of ethanol, add glycerin, and stir.

3) Dissolve β-cyclodextrin in 130 mL of pre-heated water at 60° C., and then cool the solution to room temperature.

4) Add nizatidine to the step 3) solution.

5) Dissolve sodium citrate and citric acid in 20 mL of water.

6) Dissolve sodium saccharin and sodium chloride in 25 mL of water.

7) Add the liquid mixture of steps 2), 4), 5) and 6) to step 1), and mix.

8) Add the bubble gum flavor, Prosweet, and mint flavor to the remaining ethanol, and stir.

9) Add the liquid mixture of step 8) to step 7) and stir.

10) Dilute to the final volume of 500 mL with water and mix.

11) Filter and fill the final solution of step 10) into bottles.

### EXAMPLE 2

### Pharmaceutical Solution Formulation of Nizatidine 15 mg/mL with Hydroxypropyl Methylcellulose

| Ingredient | Grams/600 mL |
|---|---|
| Nizatidine | 9 |
| Hydroxypropyl methylcellulose | 3 |
| E5 (HPMC) | |
| Methylparaben | 1.08 |
| Propylparaben | 0.12 |
| Citric acid (anhydrous) | 0.18 |
| Sodium citrate dihydrate | 1.56 |
| Sodium saccharin | 3 |
| Glycerin | 54 |
| Sodium chloride | 1.8 |
| Sucrose | 120 |
| Prosweet | 3 |
| Natural and artificial bubble gum flavor | 0.6 |
| Water | q.s. to 600 mL |

Formulation pH range: 6-7.

Manufacturing process: similar to that of Example 1, except that hydroxypropyl methylcellulose E5 (HPMC) is used instead of hydroxypropyl-β-cyclodextrin, and there is no ethanol.

### EXAMPLE 3

### Pharmaceutical Solution Formulation of Nizatidine 15 mg/mL with Xanthan Gum

| Ingredient | Grams/500 mL |
|---|---|
| Nizatidine | 7.5 |
| Xanthan gum | 0.4 |
| Methylparaben | 0.9 |
| Propylparaben | 0.1 |
| Citric acid (anhydrous) | 1.05 |
| Sodium citrate dihydrate | 0.03 |
| Sodium saccharin | 2.5 |
| Glycerin | 45 |
| Sodium chloride | 1.5 |
| Sucrose | 200 |
| Ethanol | 5 |
| Prosweet | 5 |
| Natural and artificial bubble gum flavor | 0.3 |
| Mint flavor | 0.25 |
| Water | q.s. to 500 mL |

Formulation pH range: 6-7.

Manufacturing process: similar to that of Example 1, except that xanthan gum is used instead of hydroxypropyl-β-cyclodextrin.

### EXAMPLE 4

### Pharmaceutical Solution Formulation of Nizatidine 15 mg/mL with Sodium Alginate

| Ingredient | Grams/200 mL |
|---|---|
| Nizatidine | 3 |
| Sodium alginate | 2.4 |
| Methylparaben | 0.36 |
| Propylparaben | 0.04 |
| Citric acid (anhydrous) | 0.42 |
| Sodium citrate dihydrate | 0.012 |
| Sodium saccharin | 1 |
| Glycerin | 18 |
| Sodium chloride | 0.6 |
| Sucrose | 60 |
| Ethanol | 2 |
| Artificial sweetness enhancer | 2 |
| Natural and artificial bubble gum flavor | 0.12 |
| Mint flavor | 0.1 |
| Water | q.s. to 200 mL |

Formulation pH range: 6-7.

Manufacturing process: similar to that of Example 1, except that sodium alginate is used instead of hydroxypropyl-β-cyclodextrin.

### EXAMPLE 5

### Pharmaceutical Solution Formulation of Nizatidine 15 mg/mL with Hydroxyethyl Cellulose

| Ingredient | Grams/1000 mL |
|---|---|
| Nizatidine | 15 |
| Hydroxyethyl cellulose (HEC) | 2 |
| Methylparaben | 1.8 |
| Propylparaben | 0.2 |
| Citric acid (anhydrous) | 2.1 |
| Sodium citrate dihydrate | 6 |
| Saccharin sodium | 5 |
| Glycerin | 90 |
| Sodium chloride | 3 |
| Sucrose | 400 |
| Ethanol | 10 |
| Prosweet | 10 |
| Artificial bubble gum flavor | 0.6 |
| Mint flavor | 0.5 |
| Water | q.s. to 1000 mL |

Formulation pH range: 6-7.

Manufacturing process: similar to that of Example 1, except that hydroxyethyl cellulose (HEC) is used instead of hydroxypropyl-β-cyclodextrin.

The solution is filled into amber-colored 480 mL polyethylene terephthalate (PET) bottles, closed with child resistant closures and stored at 40° C.±2° C. and 65%±5% relative humidity conditions for 3 months, and is analyzed at intervals to evaluate the extent of impurity formation and formulation component content. Impurity content values below are percentages of the label drug content, and assay values are percentages of the original content.

| | Content | | | |
|---|---|---|---|---|
| | Initial | 1 Month | 2 Months | 3 Months |
| Impurity | | | | |
| Impurity A | 0.01 | 0.01 | 0.01 | 0.02 |
| Impurity B | BLD | BLD | BLD | BLD |
| Impurity C | 0.11 | 0.20 | 0.36 | 0.61 |
| Impurity D | BLD | 0.23 | 0.56 | 1.08 |
| Impurity E | BLD | 0.20 | 0.38 | 0.40 |
| Impurity F | BLD | BLD | BLD | BLD |
| Impurity G | 0.07 | 0.10 | 0.11 | 0.12 |
| Impurity H | BLD | BLD | BLD | BLD |
| Impurity I | BLD | BLD | BLD | BLD |
| Impurity J | BLD | BLD | BLD | BLD |
| Impurity K | BLD | 0.09 | 0.31 | 0.53 |
| Impurity X | BLD | 0.14 | 0.46 | 1.06 |
| Impurity Y | 0.10 | 0.22 | 0.18 | 0.19 |
| Impurity Z | BLD | BLD | BLD | BLD |

Assay

| | | | | |
|---|---|---|---|---|
| Component | | | | |
| Nizatidine | 100.6 | 97.9 | 97.4 | 93.6 |
| Methylparaben | 99.2 | 95.6 | 91.2 | 87.3 |
| Propylparaben | 95.3 | 95.4 | 93.6 | 92.1 |

BLD = Below the limit of detection.

The levels of impurities for the formulation are found to be within acceptable limits.

We claim:

1. A pharmaceutical solution comprising nizatidine or a salt thereof and a taste-masking or taste-enhancing agent, and having a pH in the range of about 6 to about 7.

2. The pharmaceutical solution of claim 1, wherein nizatidine or salt thereof is present at a concentration of about 0.5 mg/mL to about 30 mg/mL.

3. The pharmaceutical solution of claim 1, wherein a weight ratio of nizatidine or salt thereof to the taste-masking agents is about 1:0.01 to about 1:1-5.

4. The pharmaceutical solution of claim 1, wherein a taste-masking agent comprises a cyclodextrin or derivative thereof, glycerin, xanthan gum, carrageenan, tragacanth, guar gum, pectin, carboxymethyl cellulose, hydroxyethyl cellulose, hydroxypropyl methylcellulose, methylcellulose, microcrystalline cellulose, a carboxymethyl cellulose blend, or any mixtures thereof.

5. The pharmaceutical solution of claim 1, wherein a taste-masking agent comprises hydroxypropyl-β-cyclodextrin.

6. The pharmaceutical solution of claim 1, wherein a taste-masking agent comprises xanthan gum.

7. The pharmaceutical solution of claim 1, wherein a taste-masking agent comprises hydroxyethyl cellulose or hydroxypropyl methylcellulose.

8. The pharmaceutical solution of claim 1, wherein a taste-masking agent comprises sodium alginate, with the proviso that a weight ratio of nizatidine or salt thereof to sodium alginate is about 1:0.8 to about 1:2-5.

9. The pharmaceutical solution of claim 1, wherein a taste-enhancing agent comprises citric acid, sodium citrate dihydrate, saccharin sodium, sodium chloride, sodium hydroxide, sodium carbonate, sodium bicarbonate, an artificial sweetness enhancer, or any mixtures thereof.

10. A method of treating duodenal ulcer or endoscopically diagnosed esophagitis, including erosive and ulcerative esophagitis, and associated heartburn due to GERD or active benign gastric ulcer, comprising administering a pharmaceutical solution of claim 1.

11. A pharmaceutical solution comprising about 15 mg/mL of nizatidine and a taste-masking agent comprising a cyclodextrin or derivative thereof, glycerin, xanthan gum, carrageenan, tragacanth, guar gum, pectin, a carboxymethyl cellulose, a hydroxyethyl cellulose, a hydroxypropyl methylcellulose, a methylcellulose, a microcrystalline cellulose, a carboxymethyl cellulose blend, or any mixtures thereof, and having a pH value about 6 to about 7.

12. The pharmaceutical solution of claim 11, wherein a weight ratio of nizatidine or salt thereof to taste-masking agent is about 1:0.01 to about 1:1-5.

13. The pharmaceutical solution of claim 11, wherein a taste-masking agent comprises hydroxypropyl-β-cyclodextrin.

14. The pharmaceutical solution of claim 11, wherein a taste-masking agent comprises xanthan gum.

15. The pharmaceutical solution of claim 11, wherein a taste-masking agent comprises hydroxyethyl cellulose or hydroxypropyl methylcellulose.

16. The pharmaceutical solution of claim 11, further comprising a taste-enhancing agent.

17. The pharmaceutical solution of claim 11, further comprising a taste-enhancing agent comprising citric acid, sodium citrate dihydrate, saccharin sodium, sodium chloride, sodium hydroxide, sodium carbonate, sodium bicarbonate, an artificial sweetness enhancer, or any mixtures thereof.

18. A pharmaceutical solution comprising about 15 mg/mL of nizatidine and a taste-masking agent comprising sodium alginate, with the proviso that a weight ratio of nizatidine to sodium alginate is about 1:0.8 to about 1:2-5.

19. The pharmaceutical solution of claim 18, further comprising a taste-enhancing agent.

20. The pharmaceutical solution of claim 18, further comprising a taste-enhancing agent comprising citric acid, sodium citrate dihydrate, saccharin sodium, sodium chloride, sodium hydroxide, sodium carbonate, sodium bicarbonate, an artificial sweetness enhancer, or any mixtures thereof.
